(19) United States
(12) Patent Application Publication
(10) Pub. No.: US 2003/0003144 A1
(43) Pub. Date: Jan. 2, 2003

(54) Sustained release formulations for nifedipine, dextromethorphan, and danazol

(76) Inventor: Brian C. Keller, Antioch, CA (US)

Correspondence Address: Bruce D. Grant, Morrison & Foerster LLP, Suite 500, 3811 Valley Centre Drive, San Diego, CA, 92130-2332, US

(21) Appl. No.: 10/136,957

(22) Filed: May 1, 2002

Related U.S. Application Data
(60) Provisional application No. 60/287,992, filed on May 1, 2001.

(51) Int. Cl. A61K 9/127; A61K 9/48; A61K 9/64

(52) U.S. Cl. 424/456; 514/356; 514/282; 424/450

(57) ABSTRACT

Disclosed herein are sustained release formulations of nifedipine and dextromethorphan that are compatible with a soft elastic gelatin capsule and a two-piece hard shell gelatin capsule. It has been discovered that specific lipids in the formulations can spontaneously form multilamellar liposomes upon introduction of the formulation to an aqueous environment. These spontaneously formed liposomes are stable under conditions that simulate the environment of the stomach and upper small intestine. The formulations can be administered orally, intra-ocularly, intranasally, rectally, or vaginally.

### CROSS-REFERENCE TO RELATED APPLICATION

This application claims the benefit of the May 1, 2001 filing date for U.S. Application No. 60/287,992, entitled “Sustained Release Formulations for Nifedipine and Dextromethorphan,” naming Brian C. Keller as an inventor. This application is incorporated herein by reference.

### FIELD OF INVENTION

The invention relates to the field of liposome drug delivery systems.

### BACKGROUND

The therapeutic effect of an administered substance is usually directly related to the quantity and rate at which the substance reaches the bloodstream. There are many factors that affect the ability of the substance to reach the systemic circulation, including the site of entry into the body, the physical form of the substance, the design of the formulation of the product, various physicochemical properties of the compound and the excipients, and control and maintenance of the location of the substance at the proper absorption site.

Although oral delivery of a therapeutic substance is a common form of delivery because of convenience and ease of administration, it is not the most reliable route of administration and can often be inefficient and erratic. Factors that influence absorption, and thus the ability of the substance to reach the bloodstream, of an orally administered substance are related to the physicochemical properties or the substance, the physiologic factors in the gastrointestinal tract, and the variables in the dosage form. Conventional oral dosage forms consist of solutions, suspensions, powders, two-piece gelatin capsules, soft gelatin capsules, compressed tablets, and coated tablets. It is generally the case that gastrointestinal absorption is most rapid with solutions and slower with coated tablets. Liquid dosage forms are generally absorbed more quickly than solid forms because dissolution is not a rate determining step in the absorption process.

It is the object of drug delivery technology to design a dosage form that optimizes effectiveness, maximizes drug reliability and maximizes safety of the delivered compound. Oral dosage forms improved in the late 1940's and early 1950's with the introduction of sustained-release technology. The principle benefit of this new type of delivery system was to improve drug performance by increasing the duration of drug action and reducing the dosing interval required to achieve a therapeutic effect. Controlled-drug delivery technology was developed in the late 1960's. The principle benefit of this technology is to control the rate of dissolution from the solid dosage form to enhance bioavailability, improve safety, and decrease the dosing interval. Within the last twenty years a newer concept in oral drug delivery technology has been developed and is referred to as a therapeutic system. The essential component of the therapeutic system is the incorporation of advanced engineering controls that release drug from the dosage form at appropriate times in response to stimuli, e.g., preprogrammed wax matrix.

Capsules are a convenient and popular solid dosage form used for drugs, vitamins and nutritional supplements worldwide. The drug substance is enclosed within gelatin walls of the capsule, which can be either a two piece hard shell or a soft shell (also known as the soft elastic capsule). The soft elastic capsule (SEC) is a soft, globular, gelatin shell somewhat thicker than that of hard gelatin capsules. The gelatin is plasticized by the addition of glycerin, sorbitol, or a similar polyol. The greatest advantage of soft gel capsule over two piece gelatin capsules is that soft gels can encapsulate liquids, semiliquids, and pastes due to the manufacturing process which hermetically seals the two halves together. There are several manufacturing processes by which soft gel capsules are made, those include the plate process, the rotary die process, the Norton capsule machine and Accogel, or Stem machine. A newer technology allows a two-piece gelatin capsule to tolerate liquids, semiliquids and pastes by sealing the upper and lower pieces together to prevent leakage of encapsulated material.

Liposomes are microscopic, three-dimensional lipid vesicles, made up of a phospholipid bilayer membrane that surrounds and separate an aqueous compartment. The discovery of liposomes has been credited to Alec Bangham, a British biologist and physician, who first described swollen lipid particles in the early 1960's. (Bangham A., et al, J. Mol Biol., 13, 238 (1965)). However there is evidence of the observation of multilamellar liposomes dating back to 1911. (Lasic, D., Liposomes (1993)). Two decades after Bangham and his colleagues described their discovery the field of liposome science began to take hold, and the pharmaceutical and pharmacological rational that justifies the use of liposomes as drug carriers was being put into practice. Today, the medical applications of liposomes range widely from systemic anticancer therapy to enhancing topical anesthesia and gene delivery.

Oral administration of liposomes first began in the mid 1970's. The attributes of phospholipid based liposomes were well known at the time. Medical researchers believed that this would be an ideal application to potentially enhance gastrointestinal absorption, protect the encapsulated ingredient from metabolic degradation and perhaps release the encapsulate slowly, thus providing a form of sustained release. Early studies showed that liposome encapsulated drugs were better absorbed than non-liposome encapsulated or “free” drug. In addition to drug molecules, proteins, peptides and enzymes were delivered orally with liposomes. In an attempt to develop an oral treatment for hemophilia with blood clotting factor VIII, a novel technique was developed which made possible high-yield entrapment of Factor VIII in a liposome. (Gregoridias, G. et al., J. Microencap., 1(1): 33-45 (1984)). Liposomal encapsulated Factor VIII was administered to patients orally and was absorbed from the intestines. (Sakuragawa N., Thrombosis Research 38(6): 681-5 (1985)). Early enthusiasm with liposome encapsulated insulin showed that small but significant amount of insulin could reach the circulation (Woodly, J F, Critical Rev Ther. Drug Carrier Sys. 2(1): 1-18 (1985)). Significant antibody response was elicited after oral administration of liposome-entrapped snake venom (enzymes and peptides) compared to no response from free venom. (New, R R, Toxicon. 23(2): 215-9 (1985)).

More recently, feasibility of oral liposomes for a variety of therapeutic uses has been demonstrated. Increased bioavailability of liposomally encapsulated superoxide dismutase (Regnault C., et al., Biopharm & Drug Disp 17,165-174 (1996)) a potent antioxidant used in the treatment of radiation-induced fibrosis, which is poorly absorbed orally, from 14% (free) to 57% with liposomes having ceramides. Hypocalcemia was observed one hour after the administration of liposomes loaded with 1 mg of calcitonin. (Arien, et al., Pharm Research 12(9): 1289-1292 (1995)). This result was surprising because liposomes were presumed to be unstable against the action of bile salts, however they were able to partially protect the peptide from enzymatic degradation. In another study, recombinant human erythropoietin (Epo), used to treat renal anemia, was encapsulated in liposomes. Bioavailability of oral Epo is poor because it is a protein and broken down in the gastrointestinal tract by proteolytic enzymes. Absorption and a long pharmacological effect and lag were observed, suggesting that liposomes were trapped in a site before entering the bloodstream, and eliciting a sustained release effect. (Maitani Y., J Pharm Sc 85(4): 440-445 (1996)).

Pharmaceutical challenges associated with administering liposomes orally include pH of the stomach, bile salts and digestive enzymes, primarily lipases. The unbuffered pH of the stomach can range from 1.5 to 2.5 and causes chemical instability of the liposome membrane surface. Bile salts can act as detergents and cause instability of the liposome bilayer by emulsification. Upon exposure to lipases and other enzymes, polar head groups or the acyl chains of the phospholipids can be cleaved and rupture the liposome vesicle.

### SUMMARY

Disclosed herein are formulations of nifedipine, dextromethorphan, and danazol that are compatible with a soft elastic gelatin capsule and a two-piece hard shell gelatin capsule capable of tolerating liquid in its interior. Such formulations have sustained release properties allowing for once daily dosing. It has been discovered that specific lipids in the formulations can spontaneously form multilamellar liposomes upon introduction of the formulation to an aqueous environment. These spontaneously formed liposomes are stable under conditions that simulate the environment of the human stomach and upper small intestine. Lipids in the liposome are often selected from PEG-12 glyceryl dioleate and PEG-12 glyceryl dimyristate. These lipids are compatible with the gelatin matrix in soft elastic gelatin capsules and two-piece hard shell gelatin capsules.

### DETAILED DESCRIPTION

Active Ingredients

Nifedipine is a known calcium channel-blocking agent used therapeutically to treat high blood pressure and angina pectoris. The conventional dosage form for this active pharmaceutical ingredient are a soft elastic gelatin capsule, also called ‘soft gel cap’, that is dosed three times daily and a film coated, sustained release tablet that is dosed once daily. It is therefore desirable to have a formulation that is administered in a soft gelatin capsule that has sustained release properties so it can be dosed once daily to increase patient, or user, compliance.

Dextromethorphan is the d-isomer of the codeine analog levorphanol; it lacks analgesic and addictive properties. It is a non-narcotic antitussive used commonly in over-the-counter cough and cold preparations. The usual dose of 15 mg to 30 mg is given every 4 to 8 hours. The existing dosage forms are liquids, syrups, lozenges and capsules. It would be highly beneficial to create a extended release soft gel dosage form because patients prefer this dosage form when compared to liquids, syrups, lozenges and capsules as they are easier to take and a sustained release formulation would require less frequent dosing intervals.

Danazol is a synthetic androgen derived from ethisterone. It suppresses the pituitary-ovarian axis by inhibiting the output of pituitary gonadotropins and depresses output of both follicle-stimulating hormone (FSH) and lutenizing hormone (LH). Danazol appears to exert its inhibitory effect by binding receptors of gonadal steroids at target organs. Danazol will decease IgG, IgM and IgA levels, as well as phospholipid and IgG isotope auto antibodies. Danazol has been used in the treatment of endometriosis by altering the normal and ectopic endometrial tissue so that it becomes inactive and atrophic. Danazol is metabolized hepatically and undergoes significant first pass metabolism. Blood levels of danazol do not typically increase with increased oral doses.

Formulation and Encapsulation

Formulations described herein can form liposomes upon contact with an aqueous environment. Liposomes, which are microscopic lipid vesicles having one or more lipid bilayers, have been used in drug delivery systems for topical, intravenous, inter-oral, and subcutaneous administration. Liposomes having one layer are unilamellar vesicle, and liposomes having multiple bilayers are multilamellar vesicle, where multilamellar vesicles may contain aqueous zones between each bilayer in a multilamellar vesicle. Liposomes can encapsulate both lipophilic and hydrophilic substances as the lipids in the liposome are amphiphillic, having a polar head oriented toward the outside of the vesicle and a lipophilic tail oriented toward the center of the vesicle. Thus, water soluble components can be entrapped in water between bi-layers and the lipid soluble material is entrapped in the bi-layers themselves.

Encapsulated components can be released from liposomes as each layer of the liposome is disrupted by enzymatic action within the digestive system of a subject. This phenomenon provides sustained release of an active ingredient as disruption occurs layer-by-layer to the center of the liposome.

Formulations described herein may comprise any bilayer forming lipid, including phospholipids, sphingolipids, glycosphingolipids, and ceramides. Liposomes can be mechanically stabilized using certain phospholipids, e.g., phospholipon 90H, and cholesterol at an optimum molar ratio of 2:1. The optimum ratio is expected to vary with the specific phospholipid selected. This stability can protect the liposome from gastrointestinal degradation. The lipid is sometimes glyceryl dioleate or glyceryl dimyristate, and often the lipid is conjugated with polyethylene glycol (PEG), the latter being referred to herein as a “PEG-lipid.” Many forms of PEG are known in the art and many procedures for conjugating PEG to a lipid are also known. PEG-12 is often conjugated to the lipid. The formulation may include a PEG-lipid in an amount between about 50% to about 95% by weight, often about 80% to about 95% by weight. The formulation may include cholesterol in an amount between about 0.001% to about 5% by weight, often about 0.001% by weight to about 1% by weight, and sometimes about 1% by weight.

The formulation may be a preliposome mixture that forms liposomes upon contact with an aqueous environment forms liposomes. Often, the lipids described herein will form liposomes spontaneously upon contact with an aqueous environment. As used herein, the term “spontaneously” refers to a time period for liposome formulation of less than thirty seconds, and often less than fifteen seconds. Liposomes formed by the formulations described herein can be between about 20 nm to about 1000 nm in diameter. These liposomes can be rehydrated, dehydrated, partially hydrated or fully hydrated. The formulation in the capsule often includes water in an amount between about 0.5% and 10% by weight, and sometimes in an amount of about 5% by weight.

The content of active ingredient may vary in the liposome formulation. Nifedipine is often formulated in an amount between about 1% by weight to about 20% by weight, and sometimes in an amount of about 5%. Dextromethorphan is often formulated in an amount between about 1% by weight and 10% by weight, and sometimes in an amount of about 5% by weight. Danazol is often formulated in an amount between about 5% by weight to about 50% by weight, sometimes in an amount between about 10% by weight to about 30% by weight, and sometimes in an amount of about 20% by weight.

The formulation optionally includes a surfactant in an amount of about 0.001% to about 5% by weight, often including about 0.001% by weight to about 1% by weight surfactant, and sometimes about 1% by weight surfactant. The surfactant is often dipalmitoyl phosphatidylcholine (DPPC). In addition, the formulation optionally includes an organic solvent in an amount between about 0.001% and about 5% by weight, sometimes including about 0.001% to about 1% by weight organic solvent, where the organic solvent is often benzyl alcohol. Also, the formulation optionally includes a microorganism inhibitor, many of which are known in the art. The microorganism inhibitor is often potassium sorbate, which is known to inhibit the growth of yeast and mold. Potassium sorbate may be included in the formulation in an amount between about 0.001% to about 5% by weight, often in an amount of about 0.5% by weight. Also, the formulation may optionally include a lipophilic compound, such as vitamin E, which can be included in an amount between about 0.001% by weight and about 5%, and often in an amount of about 5% by weight.

Although certain chemical and stearic modifications can be made to liposomes to enhance stability, the incorporation of a fluid liposome dispersion into a gelatin based capsule can improve stability, provide a convenient dosage form, and assist in sustained release characteristics of the liposome. The capsule can be a soft gel capsule capable of tolerating a certain amount of water, a two piece capsule capable of tolerating a certain amount of water or a two piece capsule where the liposomes are preformed then dehydrated.

Gelatin capsules have a low tolerance to water on their interior and exterior. Water tolerance of a soft gel capsule is about 10% on the interior. Some liposome formulations can require from about 60% to 90% water to maintain liposome structure. Formulations described herein provide liposomes from components in a capsule having a low water content. By generating liposomes in a low aqueous system, liposomes are able to encapsulate biologically active material with limited exposure of the capsule lining to water. The concentration of water in the formulation does not typically exceed the water content tolerance of the capsule in which the formulation is loaded. The capsule often can tolerate water in the amount of 0.5% and about 20% by weight, and sometimes in an amount of about 15% to about 20% by weight.

Capsulation of liposomes into a gelatin shell improves liposome stability because it is protected from exposure to the air and thus oxidation, thereby increasing shelf-life of the formulation. Capsulation may also protect the liposome-drug complex from the low pH of the stomach, emulsification from bile salts and degradation of the liposomes and the drug substance by digestive enzymes. This protection can be further enhanced when the outer shell of the capsule is coated with a polymer like hydroxyethylmethyl cellulose propylethyl acetate, or hydroxypropylmethylcellulose propylethyl thalami.

Administration

The liposome-capsule unit containing biologically encapsulated material can be administered orally to a subject, administered as a topical unit-of-use application, or administered via other routes of application such as intra-ocular, intranasal, rectal, or vaginal administration. Danazol formulations often form liposomes spontaneously, which then may be enrobed into a soft gelatin capsule. This type of danazol formulation may be used as a vaginal suppository for preventing or minimizing endometriosis, which would allow the danazol to be absorbed transmucosally into the blood stream and avoid the first pass hepatic metabolism.

Oral administration of liposomes was typically provided by intubation directly into the small intestine, to the back of the throat by a gavage syringe, or by dropper directly into the mouth. These modes of administration are impractical because they can be messy, provide an inaccurate dose, and are difficult for patients to handle. In addition many biologically active ingredients have a bitter, astringent, and unpleasant taste that is unpalatable and difficult to mask.

Liposomes in a capsule dosage form provide a convenient, easy to manage unit-of-use which can be more easily handled by the patient than the usual liquid form of a liposome preparation. An easy to administer dosage form, such as a capsule, leads to increased compliance by the patient. Noncompliance is disturbingly common. Over one-half of the 1.6 billion prescriptions written annually in the U.S. are taken incorrectly, and 30-50% of the prescribed medications fail to produce their intended results. The economic consequences of medication noncompliance is in excess of $100 billion annually. A significant barrier to compliance is regimen complexity. Reduction of regimen complexity includes use of convenient dosing formulations. It is estimated that 50% of the American population don't like taking oral liquids. By administering a liposome in a capsule, certain compliance issues are overcome. There has been very little discussion or development of an oral dosage form for liposomes up until now and there are few or no commercial oral liposome dosage forms available.

The following examples are intended to illustrate but not to limit the invention.

| | |
|---|---|
| Ingredient | Concentration |

Example 1
Nifedipine Formulation

| | |
|---|---|
| Nifedipine 60 mg in a 750 mg soft gel capsule: | |
| Dipalmitoyl Phosphatidylcholine (DPPC) | 2% |
| Cholesterol | 0.1% |
| Nifedipine | 8% |
| Purified water | 4% |
| Potassium sorbate | 0.2% |
| Vitamin E | 1.0% |
| PEG-12 Glyceryl Dioleate | 84.7% |

Example 2
Nifedipine Formulation

| | |
|---|---|
| Nifedipine 90 mg in a 750 mg soft gel capsule: | |
| PEG-12 Glyceryl Dimyristate | 82.95% |
| Cholesterol | 0.5% |
| Nifedipine | 12% |
| Purified water | 4% |
| Benzyl alcohol | 1% |

Example 3
Nifedipine Formulation

| | |
|---|---|
| Nifedipine 30 mg in a 550 mg soft gel capsule: | |
| PEG-12 Glyceryl Dioleate | 90.1% |
| Cholesterol | 0.5% |
| Nifedipine | 5.4% |
| Purified water | 3% |
| Benzyl alcohol | 1% |

Example 4
Dextromethorphan Formulation

| | |
|---|---|
| Dextromethorphan 30 mg in a 550 mg soft gel capsule: | |
| Dextromethorphan | 5.5% |
| DPPC | 2% |
| Cholesterol | 0.2% |
| PEG-12 Glyceryl Dioleate | 87.1% |
| Purified water | 4% |
| Potassium sorbate | 0.2% |
| Vitamin E | 1.0% |

Example 5
Dextromethorphan Formulation

| | |
|---|---|
| Dextromethorphan 30 mg in a 550 mg soft gel capsule: | |
| PEG-12 Glyceryl Dimyristate | 89.75% |
| Cholesterol | 0.75% |
| Dextromethorphan | 5.5% |
| Purified water | 3% |
| Benzyl alcohol | 1% |

Example 6
Dextromethorphan Formulation

| | |
|---|---|
| Dextromethorphan 30 mg in a 550 mg two-piece hard | |
| shell gelatin capsule: | |
| PEG-12 Glyceryl Dioleate | 89.8% |
| Cholesterol | 0.5% |
| Purified water | 4.0% |
| Potassium sorbate | 0.2% |
| Dextromethorphan | 5.5% |

Example 7
Danazol Formulation

| | |
|---|---|
| Danazol 200 mg in a 1400 mg soft gel capsule | |
| suppository: | |
| PEG-12 Glycerol Dioleate | 9.7% |
| Danazol | 14.3% |
| Purified water | 4.0% |
| Benzyl alcohol | 1.0% |
| Vitamin E | 1.0% |

Example 8
Danazol Formulation

| | |
|---|---|
| Danazol 400 mg in a 1800 mg soft gel capsule | |
| suppository | |
| PEG-12 GLycerol Dioleate | 72.6% |
| Danazol | 22.2% |
| Potassium sorbate | 0.2% |
| Purified water | 4.0% |
| Vitamin E | 1.0% |

### EXAMPLE 8

### Determination that Formulations Spontaneously Form Liposomes

The formulations described above spontaneously form liposomes in a solution of hydrochloric acid having a pH between about 1 and about 2. The hydrochloric acid solution simulates the environment of the human stomach and upper small intestine. It has been demonstrated by cryo-electron microscopic observation that these liposomes are stable in the hydrochloric acid solution for at least one hour. These studies demonstrate that the formulations will likely carry an active ingredient past the stomach and the upper small intestine for delivery in the intestine where it can be absorbed.

Modifications may be made to the foregoing without departing from the basic aspects of the invention. Although the invention has been described in substantial detail with reference to one or more specific embodiments, those of skill in the art will recognize that changes may be made to the embodiments specifically disclosed in this application, yet these modifications and improvements are within the scope and spirit of the invention, as set forth in the claims which follow. All publications or patent documents cited in this specification are incorporated herein by reference as if each such publication or document was specifically and individually indicated to be incorporated herein by reference.

Citation of the above publications or documents is not intended as an admission that any of the foregoing is pertinent prior art, nor does it constitute any admission as to the contents or date of these publications or documents. U.S. patents and other publications referenced herein are hereby incorporated by reference.

1. A formulation comprising nifedipine, dextromethorphan, or danazol and one or more PEG-lipids in a soft elastic gelatin capsule or a two-piece hard shell gelatin capsule capable of tolerating liquid in its interior.

2. The formulation of claim 1, wherein the one or more PEG-lipids spontaneously form multilamellar liposomes upon introduction of the formulation to an aqueous environment.

3. The formulation of claim 2, wherein the aqueous environment is acid conditions in the human stomach or upper small intestine.

4. The formulation of claim 1, wherein the nifedipine is in an amount between about 1% by weight to about 20% by weight.

5. The formulation of claim 4, wherein the nifedipine is in an amount of about 5%.

6. The formulation of claim 1, wherein the dextromethorphan is in an amount of between about 1% by weight and 10% by weight.

7. The formulation of claim 6, wherein the dextromethorphan is an amount of about 5% by weight.

8. The formulation of claim 1, wherein the danazol is in an amount between about 5% by weight to about 50% by weight.

9. The formulation of claim 8, wherein the danazol is an amount between about 10% by weight to about 30% by weight.

10. The formulation of claim 1, wherein the PEG-lipid is in an amount between about 50% by weight to about 95% by weight.

11. The formulation of claim 1, wherein the PEG-lipid is in an amount between about 80% by weight to about 95% by weight.

12. The formulation of claim 1, wherein the one or more PEG-lipids are selected from the group consisting of is PEG-12 glyceryl dioleate or PEG-12 glyceryl dimyristate.

13. The formulation of claim 1 which comprises a lipophilic compound in an amount between about 0.001% by weight to about 5% by weight.

14. The formulation of claim 13, wherein the lipophilic compound is cholesterol.

15. The formulation of claim 14, wherein the cholesterol is in an amount of about 0.001% by weight to about 1% by weight.

16. The formulation of claim 13, wherein the lipophilic compound is vitamin E.

17. The formulation of claim 16, wherein the vitamin E is in an amount of about 5% by weight.

18. The formulation of claim 1 which comprises a surfactant in an amount of about 0.001% by weight to about 5% by weight.

19. The formulation of claim 18, wherein the surfactant is in an amount of 0.001% by weight to about 1% by weight.

20. The formulation of claim 18, wherein the surfactant is dipalmitoyl phosphatidylcholine (DPPC).

21. The formulation of claim 1 which comprises water in an amount between about 0.5% by weight and 10% by weight.

22. The formulation of claim 21, wherein the water is an amount of about 5% by weight.

23. The formulation of claim 1 which comprises an organic solvent in an amount between about 0.01% by weight and about 5% by weight.

24. The formulation of claim 23, wherein the organic solvent is in an amount of 0.001% by weight to about 1% by weight.

25. The formulation of claim 23, wherein the organic solvent is benzyl alcohol.

26. The formulation of claim 1 which comprises a microorganism inhibitor.

27. The formulation of claim 26, wherein the microorganism inhibitor is potassium sorbate in an amount between about 0.001% by weight to about 5% by weight.

28. The formulation of claim 27, wherein the potassium sorbate is an amount of about 0.5% by weight.

29. A method for administering a formulation of claim 1 to a subject by oral, topical, intraocular, intranasal, intrarectal, or vaginal administration.

30. The method of claim 29, wherein the formulation comprises danazol and is administered by vaginal administration.
